(19) United States
(12) Patent Application Publication
(10) Pub. No.: US 2023/0040369 A1
(43) Pub. Date: Feb. 9, 2023

(54) STRUCTURALLY FITTED TRANSCATHETER AORTIC VALVE IMPLANTATION DEVICE

(71) Applicant: NANJING SAINT MEDICAL TECHNOLOGY CO., LTD., Nanjing, Jiangsu (CN)

(72) Inventor: Chenming MA, Nanjing, Jiangsu (CN)

(21) Appl. No.: 17/758,270

(22) PCT Filed: Oct. 16, 2020
(86) PCT No.: PCT/CN2020/121635

§ 371 Date: Jun. 30, 2022

(30) Foreign Application Priority Data
Jan. 9, 2020 (CN) 202010021982.8

(51) Int. Cl. A61F 2/24

(52) U.S. Cl. CPC A61F 2/2418; A61F 2230/0034

(57) ABSTRACT

An aortic valve implantation device that is delivered through a catheter and does not depend only on friction for fixation. In this device, multiple supporting arms (50) are provided on an intermediate portion (102) of a tubular body (105). The supporting arms are “D”-shaped after fully expansion, and are affixed between a narrowest part (73) of the aorta close to the heart and a narrowest part (74) on an aortic annulus (70), so as to achieve sufficient match between the outer surfaces of the support arms (50) and surrounding tissues; Each supporting arm (50) has three landing areas (54) and two bending sections (59). This device can accurately control the position of a valve to be released on the aortic annulus (70).

The present application claims the priority of Chinese Patent No. 202010021982.8 filed on Jan. 9, 2020 with National Intellectual Property Administration, PRC, titled “STRUCTURALLY FITTED TRANSCATHETER AORTIC VALVE IMPLANTATION DEVICE”, which is incorporated herein by reference in its entirety.

### TECHNICAL FIELD

The present disclosure relates to the field of medical devices, and in particular, to a structurally fitted transcatheter aortic valve implantation device that is implantable via an approach through the aorta or through a transapical approach.

### BACKGROUND

About 300 thousand people worldwide are affected by cardiac valve diseases each year. Such diseases involve abnormal leaflet tissues, e.g., excess tissue growth, tissue degeneration or rupture, tissue hardening or calcifying, or abnormal tissue position throughout the cardiac cycle (i.e., annular dilation or ventricular reshaping), leading to dysfunction of valve, e.g., leakage or blood backflow (i.e., valve insufficiency) or resistance to forward blood flow (i.e., valve stenosis).

At present, existing transcatheter aortic valves rely on the inherent properties of the stent material and are simply secured at the position of the original aortic valve by friction. For example, Patent No. CN107890382A discloses a locatable and retrievable transcatheter aortic valve, wherein a first funnel opening structure of a valve stent is in contact with the left ventricular outflow tract and aortic annulus to serve as a support, and the valve stent has a locating rod structure configured for axial locating by securing the valve stent via friction between the lower part of the valve stent and surrounding tissue. However, due to the complexity of the pathological structures, the valve stent is positioned and secured by friction only, which may cause the valve to be pulled and pressed by the original structure after implantation and may cause valve migration, resulting in the risks of embolization, falling off or ejection, thereby causing failure of the valve implantation operation.

### SUMMARY

The invention therefore intends to provide a transcatheter aortic valve implantation device that is not solely fixed by friction. It forms structural matched with the blood vessels via a special design, accurately releases the valve at the aortic annulus, and avoids the adverse events caused by the existing fixation by friction alone, thus curing the aortic valve diseases. The invention is implemented by the following technical solutions:

The invention provides a structurally fitted transcatheter aortic valve implantation device comprising a valve stent, valve leaflets, an inner skirt and an outer skirt, wherein the valve stent is radially compressible and re-expandable so as to be implanted via a catheter device, and the valve stent comprises a tubular body having a circumference extending along a longitudinal axis; a first longitudinal end portion facing, in an implanted state, the ascending aorta side of the native aortic valve; a second longitudinal end portion facing, in an implanted state, the ventricular side of the native aortic valve; and an intermediate portion connecting the first and second longitudinal end portions with each other, wherein the tubular body has an inner circumferential surface defining an inner cavity of the tubular body and an outer circumferential surface defining an outer surface of the tubular body, the inner and outer circumferential surfaces at least extending substantially concentrically with the longitudinal axis; the first and second longitudinal end portions and the intermediate portion of the tubular body are made of a grid-like structure, a plurality of support arms are provided on the intermediate portion of the tubular body, the support arms being spaced from each other around the circumference of the tubular body, and the support arms formed directly on the tubular body and without being connected to the tubular body by welding or other mechanical means of connection; the valve leaflets are fixed on the intermediate portion of the inner cavity of the tubular body, the inner skirt is fixed at the second longitudinal end portion of the inner cavity of the tubular body and fixedly connected to the valve leaflets, the outer skirt is fixed at the second longitudinal end portion of the outer cavity of the tubular body and fixedly connected to the inner skirt, and the support arms are formed integrally, and are “D”-shaped and fixed between a narrowest part of the aorta close to the heart and a narrowest part above the aortic annulus after complete expansion, such that the outer surfaces of the support arms sufficiently match surrounding tissues; each of the support arms comprises a platform portion, an upper support arm and a lower support arm, the upper and lower support arms being formed tangentially and having smooth transition, and the platform portion being parallel with blood flow and capable of contacting the anatomical structures in the transition regions of the blood vessels and valve in a parallel manner and minimizing the effect on blood flow.

According to the aortic valve implantation device disclosed herein, when the implantation procedure is completed via transaortic or transapical approach, the support arms are expanded horizontally at the aortic annulus, move the tubule toward the ascending aorta to above the aortic annulus under a compressive force in an expanded state, and are fixed at the narrowest part of the aorta close to the heart.

According to the aortic valve implantation device disclosed herein, the upper support arm, the lower support arm and the platform portion each comprises a landing area configured for acquiring a greater tension and/or compression when matched the heart and/or vessels.

According to the aortic valve implantation device disclosed herein, among the three landing areas are provided two bending sections having the same length and a smaller width than the width of the landing areas, such that the support arms are more easily bent to form a “D”-shaped configuration. According to the aortic valve implantation device disclosed herein, the three landing areas of the support arms have equal maximum widths.

According to the aortic valve implantation device disclosed herein, the support arms are distributed equidistantly or non-equidistantly around the circumference of the tubular body.

According to the aortic valve implantation device disclosed herein, the connections between the support arms and the tubular body taper from the landing areas to the bottoms. According to the aortic valve implantation device disclosed herein, the bending sections taper from the landing areas to middle portions.

According to the aortic valve implantation device disclosed herein, the tubular body and the support arms are machined by laser cutting.

According to the aortic valve implantation disclosed herein, the tubular body comprises a plurality of grid nodes, connections among the grid nodes are grid elements, in the intermediate portion the grid nodes are divided into a first node, a second node and a third node according to positions along the axis of the tubular body, two ends of each of the support arms are connected to the first node and the second node respectively, the third node is located between the first node and the second node, and the grid elements between the first nodes and the second nodes to which the support arms are attached have a length greater than that of the grid elements between the first nodes and the second nodes to which the support arms are not attached.

According to the aortic valve implantation device disclosed herein, a minimum width between the support arms and the adjacent grid elements can receive passage of only one laser beam during laser cutting, thereby maximizing the landing area of the support arms.

It should be noted that the dimensions and/or sizes used herein for describing the valve stent generally refer to a free expanded state of the valve stent, i.e., the expanded state other than any compressed circumstance. Thus, the dimensions and/or locations in a re-expanded implanted state may be different due to the compression provided by surrounding tissues.

### Beneficial Effects of Present Disclosure:

The present disclosure has advantages that structural matched of the aortic valve implantation device in the operation process is realized through the support arm structure located on the intermediate portion of the stent tubular body, thus reducing the risks of falling off, displacement or ejection in the process of implantation and increasing the success rate of valve implantation.

### BRIEF DESCRIPTION OF THE DRAWINGS

FIG. 1 shows a structural schematic view of an aortic valve implantation device according to the present disclosure;

FIG. 2 shows a schematic view of an embodiment of the aortic valve implantation device according to the present disclosure;

FIG. 3 shows a detailed view of a support arm of the aortic valve implantation device according to the present disclosure;

FIG. 4 shows another detailed view of the support arm of the aortic valve implantation device according to the present disclosure;

FIG. 5 shows a schematic view of the overall function formed by a plurality of support arms of the aortic valve implantation device according to the present disclosure;

FIG. 6 shows a schematic view of a delivery device for the transcatheter aortic valve implantation device according to the present disclosure; and

FIG. 7 shows a schematic view of the two-dimensional machining process of the intermediate portion and the support arms of the aortic valve implantation device according to the present disclosure.

### DETAILED DESCRIPTION

The present disclosure will be further illustrated with reference to the following specific examples. It should be understood that these examples are merely intended to illustrate the present disclosure rather than limit the protection scope of the present disclosure. In addition, it should be understood that various changes or modifications may be made by those skilled in the art after reading the teachings of present disclosure, and these equivalents also fall within the protection scope of the present disclosure.

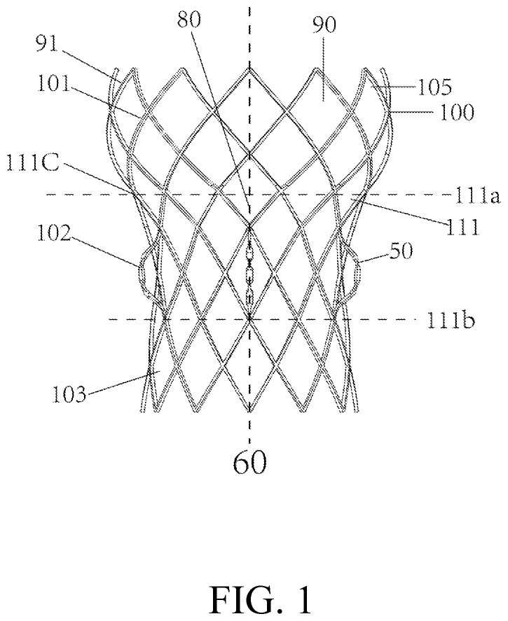

As shown in FIG. 1, a valve stent 100 comprises a tubular body 105, and the tubular body 105 consists of three portions, a first longitudinal end portion 101, an intermediate portion 102 and a second longitudinal end portion 103, which are made of a grid-like structure. The material of the tubular body 105 can be, for example, iron, nickel, aluminum, titanium, and/or alloys of these metals, and other elements. Reference numeral 111 denotes transparent artificial leaflet, each artificial leaflet 111 being connected to the inner cavity 90 of the tubular body 105. A region between two axial leaflets horizontal planes 111a and 111b that are longitudinally spaced apart from each other along the axis 60 of the tubular body 105 is a leaflet fixing region, wherein the axial leaflet horizontal plane 111a faces the first longitudinal end 101 and the axial leaflet horizontal plane 111b faces the second longitudinal end 103. The axial leaflet horizontal plane 111a spaces the first longitudinal end 101 apart from the intermediate portion 102. An artificial leaflet outflow supporting portion 111c may be provided, which is connected to a corresponding grid element 80 to fix the artificial leaflet 111 on the tubular body 105. The axial leaflet horizontal plane 111b may be located around the second longitudinal end 103. The valve stent 100 may comprise an outer skirt and an inner skirt (not shown) made of animal pericardium or artificial material.

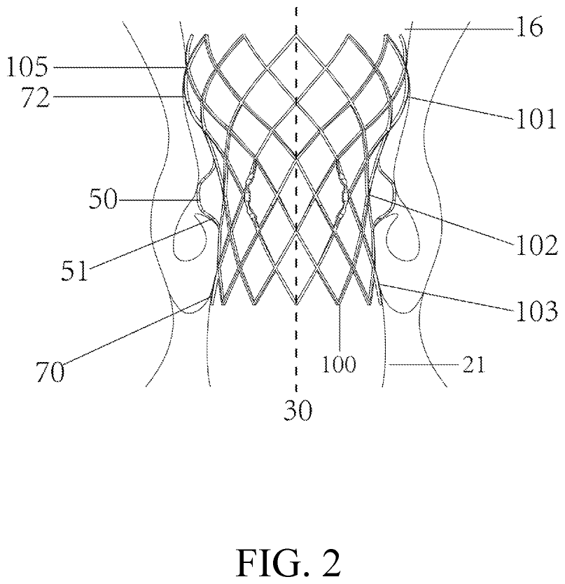

FIG. 2 shows an embodiment of the valve stent 100 for replacing a native aortic valve in a human or animal heart. That is, the valve stent 100 can be used as an artificial valve that allows blood to generally flow through the connecting channel in only one direction, in this embodiment, from the left ventricle 21 to the aorta 16, and may prevent leakage of blood in the direction from the aorta 16 to the left ventricle 21. The virtual longitudinal axis 30 is the longitudinal axis of the entire blood vessel. When the valve stent 100 is implanted, i.e., in its implanted state, the first longitudinal end portion 101 faces the aorta 16 ipsilaterally to the ascending aorta 72, and the second longitudinal end portion 103 faces the left ventricle 21 ipsilaterally to the native aortic annulus 70.

The implanted valve stent 100 is movable in its expanded state in the direction towards the aortic side 16, with the support arms 50 protruding toward the outer surface 91 of the tubular body 105. Thereby the support arms 50 move longitudinally over the native aortic annulus 70 under its radial compression. As the support arms 50 have a specific profile and are free of hooks, barbs, kinks, etc., the support arms 50 do not become entangled with the body's native tissues or cause tissue damage when moving longitudinally.

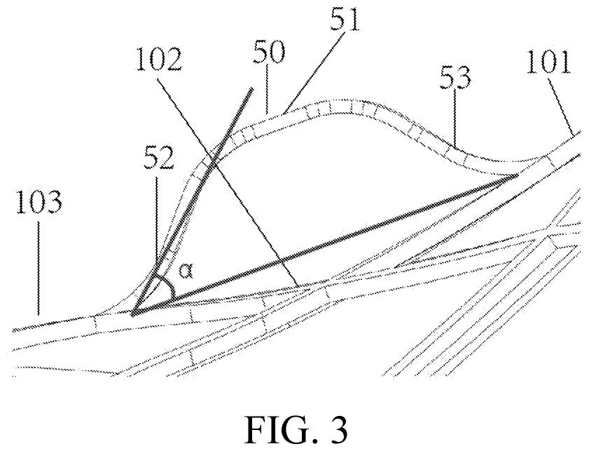

Referring to FIG. 3, the valve stent 100 may comprise a plurality of support arms 50, such as 3, 6, 9, 12 or more. The support arms 50 are integrally formed rather than being joined together by welding or other synthetic process. When the valve stent 100 expands, the support arms 50 may be circumferentially spaced from one another. The support arms 50 may be spaced from one another by equal circumferential distances, i.e., equidistantly distributed around the tubular body 105, or the support arms 50 may be spaced from one another by unequal circumferential distances. Each support arm 50 has an overall “D” shape and consists of three portions, a platform portion 51, an upper support arm 53 and a lower support arm 52. The “D” shaped design provides better integrity and anchoring function for structural matched in the transition region between the vessel and valve. The upper support arm 53, the lower support arm 52 and the platform portion 51 are centrosymmetrical. The upper support arm 53 is connected to the intermediate portion 102 towards the first longitudinal end portion 101, and the lower support arm 52 is connected to the intermediate portion 102 towards the second longitudinal end portion 103, the upper and lower support arms being formed tangentially with a smooth transition. The platform portion 51 is provided parallel to the direction of blood flow, rather than perpendicularly to the horizontal plane, and is capable of contacting the anatomical structure in the transition region of the vessel and valve in a parallel manner, minimizing the influence on blood flow.

As shown in FIG. 3, two angles are involved in the three-dimensional structure of the support arms, an upper angle and a lower angle. The upper angle and the lower angle are different in size. The lower angle a ranges from 45 degrees to 55 degrees, of which the selection is important for acquiring a D-shaped support arm with a maximum supporting force.

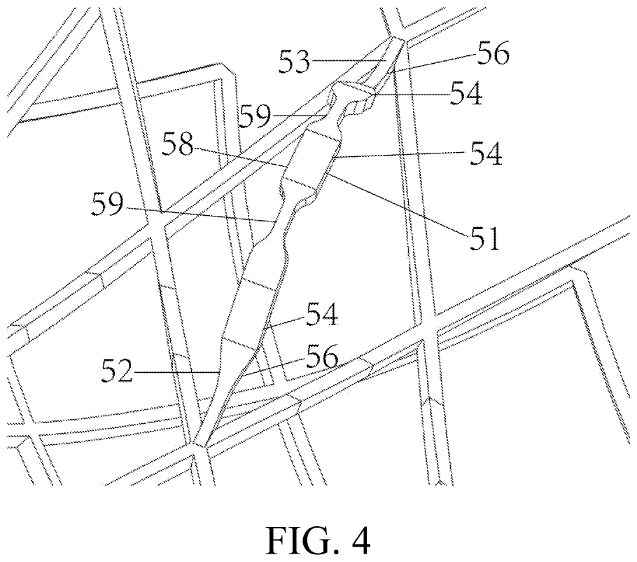

As shown in FIG. 4, the support arm 50 is further enlarged to better illustrate the formation of the structural match function. It can be seen that the upper support arm 53, lower support arm 52 and platform portion are provided with landing areas 54 for acquiring greater tension and/or compression when match occurs in the heart or vessel. Connections 56 between the support arm 50 and the implanted valve stent 100 taper from the landing areas 54 to the bottoms. Among the three landing areas 54 are provided two bending sections 59 of the same length, such that the support arms 50 are more easily bent to form a D-shaped configuration of the support arm.

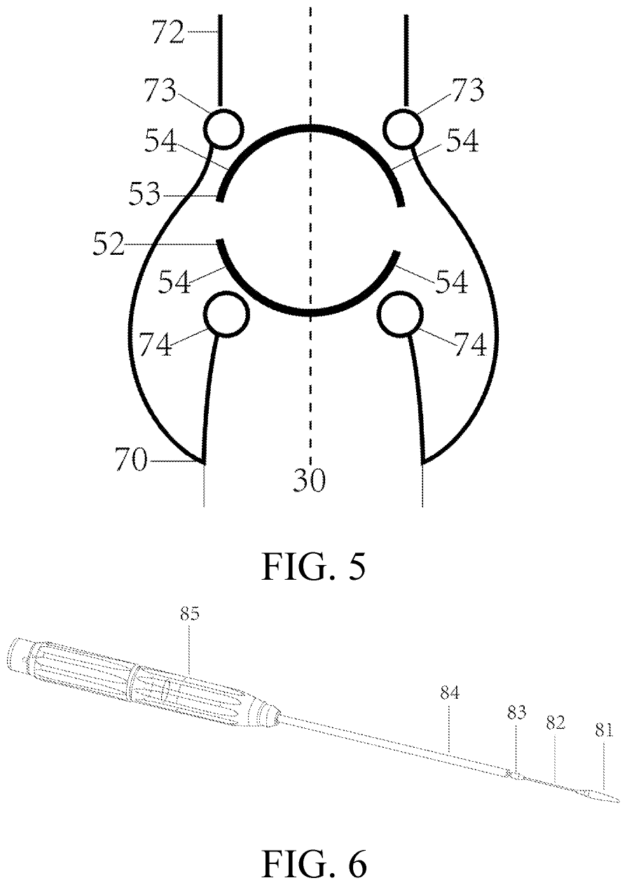

FIG. 5 schematically illustrates the function of the entirety formed by the plurality of support arms 50. The valve stent (not shown) forms an upper structural matched with the narrowest part 73 of the aorta close to the heart (e.g., the sinotubular junction, or other part of the ascending aorta) via the landing area 54 of the upper support arm 53. The valve stent (not shown) forms a lower structural matched with the narrowest part 74 above the aortic annulus (e.g., free edges of the aortic leaflet or the stenotic structures of the leaflet) via the landing area 54 of the lower support arm 52. This is clearly distinguished from existing prostheses implantation theories in the prior art relying solely on fixation by friction. The great risk of surgical failure due to fixation problems, such as displacement, falling off and ejection, is avoided by the structural matched.

Referring to FIG. 6, a schematic view of a delivery device 200 for the transcatheter aortic valve implantation device of the present disclosure is shown. The delivery device 80 of the embodiment of the present disclosure comprises: a delivery head 81, a prosthesis loading area 82, a loading site 83, a delivery catheter 84 and a rotatable grip handle 85. By the mating of the loading site 83 and the delivery head 81, the valve stent 100 is received in the prosthesis loading area 82. By slowly rotating the rotatable grip handle 85, the delivery catheter 84 will be advanced slowly (or retracted) to advance (or retract) the rotatable grip handle to complete the receiving (or release) of the valve stent from the prosthesis loading area 82.

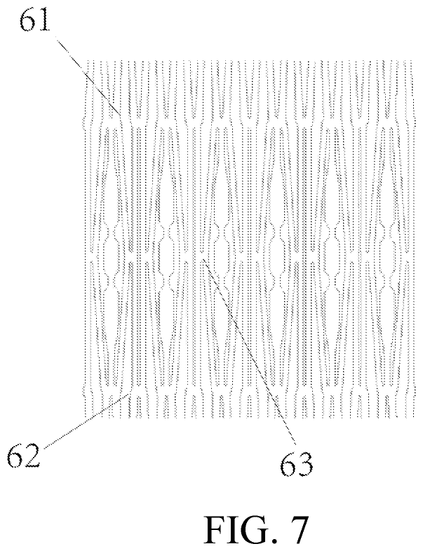

FIG. 7 is a schematic view of the two-dimensional machining process of the intermediate portion 102 and the support arms 50 of the tubular body, with the tubular body 105 and the support arms 50 being machined by laser cutting. The intermediate portion 102 of the tubular body 105 is provided with a plurality of grid nodes, connections among the grid nodes are grid elements, the grid nodes are divided into a first node 61, a second node 62 and a third node 63 according to axial positions, two ends of each of the support arms 50 are connected to the first node 61 and the second node 62 respectively, the third node 63 is located between the first node and the second node, and the grid elements between the first nodes and the second nodes to which the support arms are attached have a length greater than that of the grid elements between the first nodes and the second nodes to which the support arms are not attached.

and the grid elements between the first nodes 61 and the second nodes 62 to which the support arms are attached has a length greater than that of the grid elements between the first nodes 61 and the second nodes 62 to which the support arms are not attached. The process design allows an easier natural bending of the support arms 50 to a “D” shaped configuration when the valve stent 100 expands.

Also, a minimum width between the support arms 50 and the adjacent grid elements can receive passage of only one laser beam during laser cutting, thereby maximizing the landing area 54 of the support arms 50. A larger landing area 54 ensures a larger contact area with the anatomical structures in the transition region of the vessel and valve, enabling a desired distribution of tension and facilitating the structural matched.

The support arms 50 is such designed that the maximum widths of the three landing areas 54 are equal, which allows an easier natural bending of the support arm to a “D” shaped configuration when the valve stent 100 expends.

In the grid structure of the tubular body, different widths of the structures are designed at different grid nodes according to different radial forces.

Examples of the present disclosure have been described above. However, the present disclosure is not limited to the above examples. Any modification, equivalent, improvement and the like made without departing from the spirit and principle of the present disclosure shall fall within the protection scope of the present disclosure.

1. A structurally fitted transcatheter aortic valve implantation device comprising a valve stent, valve leaflets, an inner skirt and an outer skirt, wherein the valve stent is radially compressible and re-expandable to facilitate transcatheter implantation, and the valve stent comprises a tubular body having a circumference extending along a longitudinal axis;
a first longitudinal end portion facing, in an implanted state, the ascending aorta side of the native aortic valve;
a second longitudinal end portion facing, in an implanted state, the ventricular side of the native aortic valve;
and an intermediate portion connecting the first and second longitudinal end portions with each other, wherein the tubular body has an inner circumferential surface defining an inner cavity of the tubular body and an outer circumferential surface defining an outer surface of the tubular body, the inner and outer circumferential surfaces at least extending substantially concentrically with the longitudinal axis;
the first and second longitudinal end portions and the intermediate portion of the tubular body are made of a grid-like structure, a plurality of support arms are provided on the intermediate portion of the tubular body, the support arms are spaced from each other around the circumference of the tubular body, and the support arms are directly formed on the tubular body and without being connected to the tubular body by welding or other mechanical means of connection;
the valve leaflets are fixed on the intermediate portion of the inner cavity of the tubular body, the inner skirt is fixed at the second longitudinal end portion of the inner cavity of the tubular body and fixedly connected with the valve leaflets, the outer skirt is fixed at the second longitudinal end portion of the outer cavity of the tubular body and fixedly connected with the inner skirt, and the support arms are integrally formed, and are “D”-shaped and fixed between a narrowest part of the aorta close to the heart and a narrowest part above the aortic annulus after complete expansion, such that outer surfaces of the support arms sufficiently matched surrounding tissues;
each of the support arms comprises a platform portion, an upper support arm and a lower support arm, the upper and lower support arms being formed tangentially and having smooth transition, and the platform portion being parallel with blood flow.

2. The structurally fitted transcatheter aortic valve implantation device according to claim 1, wherein, when the implantation procedure is completed via transaortic or transapical approach, the support arms expand horizontally at the aortic annulus, move the tubular body toward the ascending aorta to above the aortic annulus under a compressive force in an expanded state, and are fixed at the narrowest part of the aorta close to the heart.

3. The structurally fitted transcatheter aortic valve implantation device according to claim 2, wherein the upper support arm, the lower support arm and the platform portion each comprise a landing area configured for acquiring a greater tension and/or compression when matched the heart and/or vessels.

4. The structurally fitted transcatheter aortic valve implantation device according to claim 3, wherein among the three landing areas are provided two bending sections having the same length and a smaller width than the width of the landing areas, such that the support arms are more easily bent to form a “D” shape configuration.

5. The structurally fitted transcatheter aortic valve implantation device according to claim 4, wherein the three landing areas of the support arms have equal maximum widths.

6. The structurally fitted transcatheter aortic valve implantation device according to claim 5, wherein the support arms are distributed equidistantly or non-equidistantly around the circumference of the tubular body.

7. The structurally fitted transcatheter aortic valve implantation device according to claim 6, wherein the connections between the support arms and the tubular body taper from the landing areas to the bottoms.

8. The structurally fitted transcatheter aortic valve implantation device according to claim 7, wherein the bending sections taper from the landing areas to middle portions.

9. The structurally fitted transcatheter aortic valve implantation device according to claim 1, wherein the tubular body and the support arms are machined by laser cutting.

10. The structurally fitted transcatheter aortic valve implantation device according to claim 1, wherein the tubular body comprises a plurality of grid nodes, connections among the grid nodes are grid elements, in the intermediate portion the grid nodes are divided into a first node, a second node and a third node according to positions along the axis of the tubular body, two ends of each of the support arms are connected to the first node and the second node respectively, the third node is located between the first node and the second node, and the grid elements between the first nodes and the second nodes to which the support arms attached has a length greater than that of the grid elements between the first nodes and the second nodes to which the support arms are not attached.

11. The structurally fitted transcatheter aortic valve implantation device according to claim 1, wherein a minimum width between the support arms and the adjacent grid elements can receive passage of only one laser beam during laser cutting, thereby maximizing the landing area of the support arms.
